(19) United States
(12) Patent Application Publication
(10) Pub. No.: US 2005/0227298 A1
(43) Pub. Date: Oct. 13, 2005

(54) Method for antigen retrieval and submersion fluid compositions for use therein

(75) Inventor: Maia Gourevitch, Amsterdam (NL)

Correspondence Address: LADAS & PARRY, 26 WEST 61ST STREET, NEW YORK, NY, 10023, US

(73) Assignee: PICKCELL LABORATORIES B.V.

(21) Appl. No.: 11/134,689

(22) Filed: May 20, 2005

Related U.S. Application Data
(62) Division of application No. 10/216,545, filed on Aug. 9, 2002.

(51) Int. Cl. G01N 33/53; G01N 33/567

(52) U.S. Cl. 435/7.2

(57) ABSTRACT

The present invention relates to a method for retrieving the antigenicity of a formaldehyde-fixed sample for immunological staining and to compositions used in such a method. A method of the invention comprises submerging a sample in a submersion fluid composition comprising an osmotically active compound and heating the said submerged sample under pressure. The present invention also discloses advantageous submersion fluid compositions for retrieving the antigenicity of a formaldehyde-fixed samples and their use.

### FIELD OF THE INVENTION

The invention relates to a method for retrieving the antigenicity of tissue sample fixed by cross-linking agents for immunological staining and to compositions used in such a method.

### BACKGROUND OF THE INVENTION

Immunohistochemical and immunofluorescent techniques characteristically involve the use of antibodies for the specific detection of antigens in tissue samples. In order to preserve the morphology of the tissue from the moment of sampling, proper fixation procedures and embedding of the sample in a rigid matrix should be employed. Routinely, tissue samples are fixed with 10% formalin (i.e., about 4% formaldehyde), embedded in paraffin and attached to a microscope slide for further immunohistochemical or immunofluorescent processing. In many instances, samples are stocked and stored until later (re)examination.

The routine procedure of using buffered formalin for fixation followed by paraffin embedding provides a well-preserved tissue infrastructure. However, formaldehyde fixation is generally not compatible with immunohistochemical staining. This is due to the fact that formaldehyde cross-links the polypeptide antigens. Such cross-linked antigens are generally no longer recognized by antibodies as used in immunohistochemical or immunofluorescent staining.

Although several alternatives to formaldehyde may be used, such as ethanol, methanol, methacarn or glyoxal, formaldehyde-based preservatives are the most widely used as they exhibit extremely powerful fixation characteristics.

In order to allow for immunohistochemical or immunofluorescent staining, the antigenicity of the sample material must be retrieved or unmasked. One method of retrieving the antigenicity of formaldehyde cross-linked proteins involves the treatment of the sample with proteolytic enzymes. This method results in a (partial) digest of the material and mere fragments of the original proteins can be accessed by antibodies.

Another method for retrieving the immunoreactivity of formaldehyde cross-linked antigens involves the thermal processing using heat or high energy treatment of the samples. Such a method is described in e.g. U.S. Pat. No. 5,244,787, wherein formaldehyde-fixed tissue preparations are submersed in water and subjected to microwave energy at temperatures sufficient to boil the water. An important problem with this method is that gas-bubbles formed during the heating of the water destroy the morphology of the tissue. In fact, relatively large holes appear in microwave-treated tissue samples from which complete nuclei seem to be removed. Further, the microwave procedure is cumbersome and the boiling of large quantities of water in a microwave oven takes substantial amounts of time.

Yet another method for retrieving antigens from formaldehyde-fixed tissues is the use of a pressure cooker, either in combination with a microwave or in the form of an autoclave, such as described in e.g. Norton, 1994. J. Pathol. 173(4):371-9 and Taylor et al. 1996. Biotech Histochem 71(5):263-70. However, these methods can also not prevent that the tissue morphology is destroyed, especially in fragile area's.

### SUMMARY OF THE INVENTION

It is an object of the present invention to provide an improved method for retrieving the immunoreactivity of antigens in tissue sample fixed by cross-linking agents while preserving the tissue morphology.

It has been found that this method enables a level of immunofluorescent staining which is comparable to that of unfixed or fresh tissue samples.

It has further been found that the use of a specific composition of a submersion fluid in combination with heating under pressure enables the retrieval of antigens fixed by cross-linking agents while maintaining tissue morphology.

In one aspect the present invention provides a method for the preparation of a tissue sample fixed by cross-linking agents for immunological staining comprising submerging said sample in a submersion fluid composition comprising an osmotically active compound and heating the said submerged sample under pressure.

In another aspect the present invention provides submersion fluid compositions comprising an osmotically active compound for use in a method of the invention.

### DESCRIPTION OF THE DRAWINGS

FIG. 1 is a microscopic image exemplifying the detection of Ki 67 antigens in human intestinal tissue. Sections of human intestinal tissue were treated according to a method of the invention by applying one cycle of heating under pressure (121° C. under a pressure of about 2 bara) of fixed sections submerged in a citrate containing submersion fluid composition according to the invention (composition B of Table 1). Ki 67, a widely used proliferation marker and nuclear antigen, was detected using monoclonal antibody MIB-1 and a 2 step immunoperoxidase detection system as described in example 3.

FIG. 2 is a microscopic image exemplifying the detection of PCNA, a widely used proliferation marker and nuclear antigen in sections of human intestinal tissue. Sections were treated according to a method of the invention by applying one cycle of heating under pressure (121° C. under a pressure of about 2 bara) of fixed sections submerged in a citrate containing submersion fluid composition according to the invention (composition B of Table 1). The antigen was detected using monoclonal antibody PC-10 and a 2 step immunoperoxidase detection system as described in example 3.

FIG. 3 is a microscopic image exemplifying the detection of CD8, a marker for killer T-cells and a cell membrane antigen in sections of human intestinal tissue. Sections were treated according to a method of the invention by applying one cycle of heating under pressure (121° C. under a pressure of about 2 bara) of fixed sections submerged in a EDTA containing submersion fluid composition according to the invention (composition I of Table 1). The antigen was detected using monoclonal antibody NCL-CD8-4B11 (Novocastra Laboratories Ltd) to CD8 and 2 step immunoperoxidase detection system as described in example 3.

FIG. 4 is a microscopic image exemplifying the detection of E-cadherin, a human cell adhesion molecule of epithelial cells and a cell membrane antigen of human cervix. Sections of human cervix were treated according to a method of the invention by applying one cycle of heating under pressure (121° C. under a pressure of about 2 bara) of fixed sections submerged in a citrate containing submersion fluid composition according to the invention (composition B of Table 1). The antigen was detected using monoclonal antibody HECD-1 and 2 step immunoperoxidase detection system as described in example 3.

FIG. 5 is a microscopic image of freshly fixed and otherwise unprocessed squamous epithelium of human cervix tissue as seen at low magnification as described in example 3 using submersion fluid composition B of Table 1.

FIG. 6 is a microscopic image of freshly fixed and otherwise unprocessed squamous epithelium of human cervix tissue as seen at high magnification as described in example 3 using submersion fluid composition B of Table 1.

FIG. 7 is a microscopic image of freshly fixed squamous epithelium tissue of human cervix that was microwave processed according to a method of the prior art as seen at low magnification as described in example 3 using submersion fluid composition B of Table 1.

FIG. 8 is a microscopic image of freshly fixed squamous epithelium tissue of human cervix that was microwave processed according to a method of the prior art as seen at high magnification as described in example 3 using submersion fluid composition B of Table 1.

FIG. 9 is a microscopic image of freshly fixed squamous epithelium tissue of human cervix that was processed by heating under pressure according to a method of the invention as seen at low magnification as described in example 3 using submersion fluid composition B of Table 1.

FIG. 10 is a microscopic image of freshly fixed squamous epithelium tissue of human cervix that was processed by heating under pressure according to a method of the invention as seen at high magnification as described in example 3 using submersion fluid composition B of Table 1.

FIG. 11-16 are microscopic images comparable to those of FIGS. 5-10, except that glandular epithelium tissue of human sigmoid was tested as described in example 3 using submersion fluid composition B of Table 1.

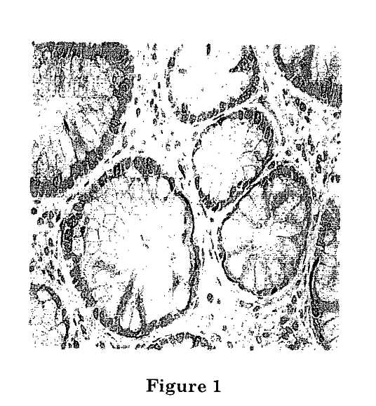

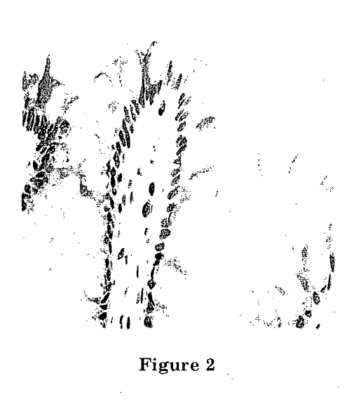

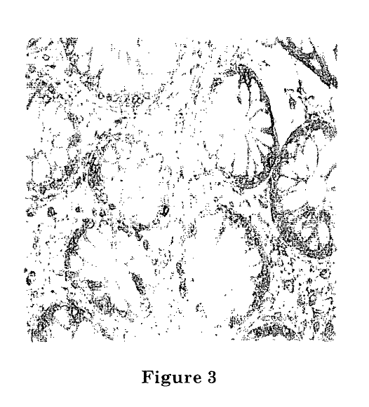

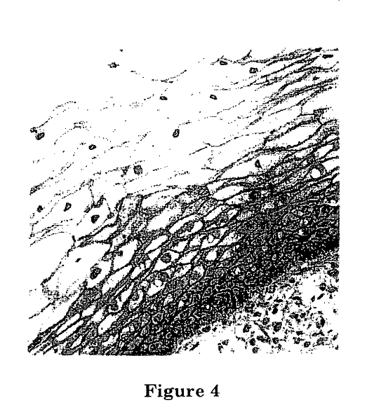

### DETAILED DESCRIPTION OF THE INVENTION

The term immunohistochemical staining as use herein is defined as the histological staining technique wherein antibodies are used to identify or mark specific cell structures or antigens within that tissue. Generally such antibodies are labelled with chromogenic labels or enzymatic labels, such as horse radish peroxidase. Also fluorescent labels may be used in such a technique in which case is referred to the term immunofluorescent staining as used herein.

A method of the invention can suitably be applied to tissue sample fixed by cross-linking agents, such as formaldehyde-fixed samples, but also samples fixed with PLP (Periodate/Lysine/Paraformaldehyde; McLean and Nakane (1974. J Histochem Cytochem, 22, 1077-1083), paraformaldehyde, Boonfix I, Boonfix II, Myrsky fixative, Bouin's solution, glutaraldehyde, zinc formalins, or other aldehydes, or other bi-functional cross-linkers can suitably be subjected to a method of the invention. Formaldehyde produces mild cross linkages when compared to other aldehyde fixatives such as glutaraldehyde.

A method of the invention can be applied to paraffin embedded tissue samples that are fixed by cross-linking agents, such as tissue biopsies from tonsils, gut, lymph nodes, prostate, cervix, liver, kidney, spinal cord, lymphoma, breast carcinoma or melanoma and the like, or mucous swabs, in order to retrieving the immunoreactivity of antigens therein. In fact, any sample fixed by cross-linking agents prior to immunopathologic or immunohistochemical or immunofluorescent examination may be used in a method of the invention. Such samples may be essentially fluidic samples, such as animal or human bodily fluids, like blood samples, but also environmental samples such as water samples. Preferably, a method of the invention is applied to samples containing cells.

A method of the invention can be applied to cells of micro-organisms fixed with cross-linking agents in order to retrieve the immunoreactivity of antigens associated with such micro-organisms. Both yeast, fungal and bacterial antigens may be retrieved by using a method of the invention, but also viral antigens may be retrieved.

Alternatively, a method of the invention may be applied to proteins fixed by cross-linking agents, such as formaldehyde-fixed proteins on a solid support, such as on a western blot, in order to retrieve their antigenicity.

Prior to subjecting samples to a method of the invention, tissue-embedding material such as paraffin may be removed by methods known in the art. Also the samples may be pretreated such as being subjected to a washing step or the like. Preferably, samples fixed by cross-linking agents are washed several times in aqueous ethanol solutions, such as 50% or 70% ethanol in water, for a period of between 30 to 200 min.

Fixed samples that are essentially fluidic such as animal or human bodily fluids fixed by cross-linking agents, but also water samples fixed by cross-linking agents are preferably washed with a buffer or a suitable other washing medium to remove essentially all formaldehyde-containing preservation fluid whereby this preservation fluid is replaced by a submersion fluid composition of the invention.

It is an aspect of a method of the invention that the sample is submerged in a submersion fluid composition comprising an osmotically active compound during the heating under pressure of the method of the invention. The fluid composition may comprise water as a carrier fluid, but preferably the carrier fluid is an aqueous buffer.

Suitable buffers for use in a submersion fluid composition of the invention include such buffers as routinely employed in immunochemistry, such as Tris-HCl, Citrate, Glycine, phosphate, PBS, HEPES, MES, MOPS, Tris-buffered saline, etc., or combinations thereof. Also, alkaline EDTA solutions may be used as a buffer in a submersion fluid composition of the invention.

The amount of buffering reagent in a submersion fluid composition of the invention may be selected in a range of between 1 mM and 1 M, preferably buffering reagents are used in an amount in a range of between 1 mM and 100 mM, more preferably of about 10 mM.

A submersion fluid composition of the invention may further comprise excipients such as blocking agents, like BSA, nonfat milk or casein, chelating agents, such as EDTA, detergents, such as Tween™, surfactants, and/or metal salts, such as salts from transition metals such as zinc or lead or salts from the alkali earth metals Na, K or Li, or other metal salts. When present, such excipients are preferably present in a submersion fluid composition of the invention in an amount of between 0.05 wt. % and 5 wt %.

It is possible that not all antigens are retrieved by using a single formulation of the submersion fluid composition of the invention. The formulation may e.g. be varied by varying the type of buffer, the type and amount of excipient and the pH. The pH at which a submersion fluid composition of the invention yields optimal antigen retrieval results depends on the reagents used therein, on the fixative and on the antigen. A submersion fluid composition of the invention is buffered to a pH that results in retrieval of the fixed antigens in a method of the invention. The pH may be selected in a range of between 4 and 10, preferably between 5 and 8, more preferably about 6.

For the retrieval of antigens in old tissue samples, i.e. that have been stored fixed for a long period of time, or for the retrieval of antigens in over-fixed samples, additional retrieval agents may be added to the submersion fluid composition. Suitable retrieval agents comprise such compounds as a guanidinium salt, preferably in the form of guanidinium thiocyanate, or urea. These compounds may be used in a submersion fluid composition in an amount of between 0.01 mM and 2 M, preferably in an amount of between 1 mM and 500 mM.

A submersion fluid composition according to the invention comprises between 1 wt. % and 99 wt. % of an osmotically active compound, based on the weight of the composition. The osmotically active compound can be selected from the group consisting of polyols or alditols, such as arabitol, dulcitol, erythritol, ethylene glycol, glycerol, inositol, lactitol, maltitol, mannitol, propylene glycol, ribitol, sorbitol, threitol and xylitol, aldoses, such as xylose, acesulfame, allose, altrose, arabinose, erythrose, fructose, galactose, glucose, gulose, idose, isomaltose, lactose, lyxose, maltose, mannose, melezitose, psicose, raffinose, rhamnose, ribose, saccharose, sorbose, stachyose, sucrose, tagatose, talose, threose, trehalose, xylose and xylulose, methylamines, such as betaine and glycerophosphorylcholine, and amino acids, such as taurine or proline or other compounds usually applied in the field of cryoprotection, such as DMSO derivatives or combinations thereof. Preferably, the osmotically active compounds are selected from the group consisting of polyols, and is more preferably glycerol.

The amount of the osmotically active compound as used in a submersion fluid composition of the invention may depend on the type of compound used, on the fixative used to fix the sample and on the antigen to be retrieved. In the case of glycerol, a preferable amount is between 5 wt. % and 75 wt. %, more preferably between 10 wt. % and 50 wt. %, even more preferably between 10 wt. % and 25 wt. %, based on the weight of the composition. In any case should a sufficient amount of osmotically active compound be provided to preserve the morphology. When, for example, guanidinium is used as a retrieval agent in a submersion fluid composition of the invention, the amount of osmotically active compound therein should be increased such as to preserve the morphology of the tissue during the heating under pressure.

A method of the invention comprises submersion of samples in a submersion fluid composition comprising an osmotically active compound and heating the thus submerged sample under pressure. Such a step may be suitably conducted in a pressure chamber such as provided by e.g. an autoclave or pressure cooker.

The heating under pressure of the invention may comprise the heating to a temperature of between 100° C. and 130° C. under a corresponding pressure of between 1.01 and 2.70 bara. The pressure that can be used in a method of the invention is very suitably steam pressure generated by heating an amount of aqueous fluid in a pressure chamber. The pressure applied is essentially chosen such that the submersion fluid wherein the samples are submerged and that is present in the pressure chamber does not boil. Preferably, heating under pressure according to the invention comprises the heating to a temperature of about 121° C. under a pressure of about 2 bara.

It is essential that either the heating or the pressurization or both are controlled during the heating of the submerged samples under pressure in a method of the invention so that boiling of the said submersion fluid is essentially prevented. The person skilled in the art can take information from steam tables to determine a suitable pressure that is to be maintained or controlled during heating of the submerged sample at a certain temperature. Also a suitable maximum temperature can be selected based on a required pressure of the steam when steam pressure is used in a method of the invention in which case the temperature is controlled.

Preferably, in a method of the invention the temperature is controlled and the steam pressure is allowed to be in equilibrium with the temperature of the aqueous fluid. In accordance therewith, the temperature of the pressure chamber is preferably controlled by controlling the temperature of the aqueous fluid provided to the pressure chamber during the heating of the submerged samples under pressure.

A formaldehyde fixed sample can be treated by a method of the invention by subjecting the submerged sample to heating under pressure for a period of between 1 second and 1 hour. Preferably the period during which the submerged sample is subjected to heating under pressure is between about 1 min and about 10 min, more preferably about 6 min.

After heating under pressure of the submerged sample in a method of the invention, the pressure is allowed to reach equilibrium with atmospheric pressure over a certain period of time. Preferably, the heat and pressure treated samples are allowed to cool slowly. More preferable, the pressurized chamber containing the treated samples is allowed to cool unforced at room temperature.

After going through a cycle of heating and cooling, a sample may be treated for an additional cycle of heating under pressure and cooling before being stained by immunohistochemical or immunofluorescent techniques.

After treating a sample for retrieval of antigenicity according to a method of the invention the sample may be stained by any suitable immunological staining technique. Such techniques are well know in the art of immuno(histo)chemistry, immunopathology and immunology and comprise staining with antibodies. Such antibodies may either be labeled with gold or silver particles, or they may be labelled with chromogenic labels such as enzymatic labels, or with luminescent labels.

A very suitable staining technique to which a sample treated according the a method of the invention may be subjected comprises a fluorescent staining. An important advantage of the present invention is that the background fluorescence or autofluorescence of the tissue sample treated according to a method of the invention is greatly reduced. Therefore, the use of immunofluorescent staining techniques for the staining of samples treated according to a method of the invention is very advantageous.

In another aspect the present invention provides submersion fluid compositions comprising an osmotically active compound for use in a method of the invention. Such a submersion fluid composition may comprise the carrier fluid described supra and an osmotically active compound as described supra. Further, a submersion fluid composition of the invention may comprise excipients and additional retrieval agents as described herein above.

The present invention will now be illustrated by the following Examples, which are in no way indented to limit the scope of the invention.

### EXAMPLE 1

### Embedding Tissues in Paraffin

Specimen Fixation

In addition to the choice of fixative, important factors for proper fixation include fixation time, temperature and pH. Fixation time will depend upon the size of the specimen. In order to achieve adequate and consistent fixation it is essential that lymphoreticular specimens be sliced to a maximum thickness of 3 mm on arrival in the laboratory. Tissue such as lymph node (3 mm slices), skin and bone marrow trephines are routinely fixed for approximately 24-48 hours at room temperature. Dense tissue such as spleen may require extended fixation. The rate of fixation can be increased by raising the ambient temperature. However this is not recommended with lymphoid tissue as it has an impaired effect on morphology. The pH of the formaldehyde solution is generally between 5 and 7, which is governed by the pH of the local water supply.

A haematoxylin and eosin (H&E) stained section is cut from each paraffin block. In addition, a Gordon and Sweet's reticulin stain is performed on all lymph node and spleen cases. After initial examination of the H&E section either additional tinctorial stains or specific panels of immunocytochemical markers are performed.

For fixation and paraffin embedding of tissues used in the present examples, tissue was cut into 4 to 7 mm blocks and placed in 3.7% formaldehyde solution in water or PBS for a period of from 4 hrs to overnight. Washed 3×50% ethanol for 1-3 hrs per wash. The fixed tissue blocks were transferred to a 50% aqueous ethanol solution for 1 hr, followed by a 2 times incubation for 1 hr each in 70% ethanol, a 2 times incubation for 1 hr each in 96% ethanol, a 2 times incubation for 1 hr each in 100% ethanol, a 2 hrs incubation in xylene, and a 2 times incubation for 2 hrs each in Paraplast Plus™ (Merck GmbH). All incubations were performed at room temperature.

### Example 2

### Coating of Microscope Glass Slides with APES and Glutaraldehyde

All paraffin embedded tissue was cut at a thickness of 3-5 μm using a Leica RM2135 microtome. The sections were floated on a warm water bath (45° C.), before being picked up onto microscope slides and allowed to drain. Sections for tinctorial staining were placed on a hot plate (56° C.) for 15 minutes before staining. Sections for immunocytochemical staining were picked up on aminopropyltriethoxysilane (APES) coated slides and dried overnight in an incubator at 37° C.

For APES coating, microscope glass slides were placed in slide chambers filled with 7.5% rosal liquid solution in demineralized water and left for 2 hrs. After that slides were rinsed for 1 hr in tap water, then for 30 min in demineralized water and left overnight at 56° C. to dry. After drying, slides were submerged in a freshly made solution of methanol with 2% APES (3-aminopropyltriethoxysilane, Sigma Cat. no. A-3648) for a period of 5 min. The slides were rinsed in methanol for 5 min, followed by a rinse with demineralized water for 5 min and were dried overnight at 37° C. After an incubation for 5 min in demineralized water containing 3% glutaraldehyde, the slides were rinsed for 5 min in demineralized water and dried overnight at 37° C. Slides were kept at room temperature until use.

### Example 3

### Immunohistochemical Staining of Paraffin Embedded Sections

Deparafinization

Sections were dewaxed by placing the slides in a Coplin jar, according to the following schedule: xylene, three changes, 5 min each; 99% (v/v) ethanol, two changes, 5 min each; methanol+0.3% H₂O₂ for 30 min (in order to block endogenous peroxidase activity); followed by a graded ethanol series of 90 (5 min), 70 (1-3 min), 50 (1-3 min) and 30% (v/v) ethanol (1-3 min); demineralized water, 5 min; and a final rinse in PBS.

Retrieval of Antigenicity.

Slides with various types of tissues were placed in a slide chamber and were submersed in different submersion fluid compositions (Table 1) in order to test their ability to retrieve antigens while preserving the morphology of the tissue. The chambers were placed in a rack inside the pressure chamber of a adapted laboratory model autoclave (Prestige Medical Series 2100), to which an amount of 125 ml of demineralized water was added. The closing lid was closed. The apparatus was heated until a temperature of 121° C. and a pressure of about 2 bara was reached inside the pressure chamber and maintained at that temperature and pressure for a period of 6 min. After that, heating was stopped and the apparatus was left to cool over a period of 2 hours to overnight without forced cooling or release of pressure. The apparatus was opened and the slide chambers were removed from the rack. De slides with the tissue sections were washed 3 times for 5 min in tap-water followed by a wash for 10 min in PBS+0.05% Tween-20.

Staining

The glass around the tissue sections was cleaned. A suitable primary antibody for detection of the antigen was applied to the tissue sections in appropriate dilution and the tissue sections were incubated with the antigen solution overnight at 4° C. in a humid chamber. The slides were washed 3 times for 10-15 min in PBS containing 0.05% Tween-20. The glass around the tissue sections was cleaned and a matching HRP conjugated secondary antibody was applied for a period of 1 hour. After binding of the secondary antibody, the slides were washed 5 times in PBS for 15 min. The glass around the tissue sections was quickly wiped dried and the staining was develop by applying freshly prepared DAB solution (0.05% DAB in 0.05M Tris-HCl (pH 7.4-7.6) with 0.03% H₂O₂ added just prior to application to the sections) to the still wet tissue sections. The sections were rinsed in tap-water and briefly stained in Mayer's hematoxylin solution for 0.5 min. For microscopic observation, the sections were washed under running tap water for 3 min, dried and mounted in Aquamount (BDH Chemicals, Dorset, England)).

TABLE 1
Tested submersion fluid compositions:

| | |
|---|---|
| A. | 0.01 M Citrate pH6.0 (adjusted with NaOH) |
| B. | 0.01 M Citrate pH6.0, 25 vol. % glycerol |
| C. | 0.01 M Citrate pH6.0, 50 vol. % glycerol |
| D. | Guanidine thiocyanate, 0.01 M pH6.0 |
| E. | Guanidine thiocyanate, 0.01 M pH6.0, 25% glycerol |
| F. | Guanidine thiocyanate, 0.01 M pH6.0, 50% glycerol |
| G. | Guanidine thiocyanate, 0.05 M pH6.0, 25% glycerol |
| H. | Guanidine thiocyanate, 0.05 M pH6.0, 50% glycerol |
| I. | EDTA, 0.01 M pH8.0, 25% glycerol |
| K. | TRIS-EDTA, 0.01 M TRIS, 0.01 M EDTA, pH9.0, 25% glycerol |

TABLE 2
Tested antibodies

| Target antigen | Species origin of specific IgGs |
|---|---|
| p53 | rabbit |
| Bad mouse | rabbit |
| mdm2 mouse | rabbit |
| Bax mouse | rabbit |
| Bcl-2 mouse | rabbit |
| Kip-1 | rabbit |
| Bag-1 mouse | rabbit |
| Bid mouse | rabbit |
| Bak mouse | rabbit |
| Bfl-1 mouse | rabbit |
| filagrin | rabbit |
| keratin 13 mouse | rabbit |
| keratin 8 mouse | rabbit |
| keratin 2 mouse | rabbit |
| keratin 5 mouse | rabbit |
| keratin 14 mouse | rabbit |
| keratin 6 mouse | rabbit |
| keratin 1 mouse | rabbit |
| keratin 10 mouse | rabbit |
| Mcl-1 mouse | rabbit |
| Kip-2 mouse | rabbit |
| Ki67 mouse epitope 1 | rabbit |
| Cadherin N | rabbit |
| Waf-1 | rabbit |
| CD34 | mouse |
| Catenin Delta | mouse |
| Cadherin N | mouse |
| Conductin | mouse |
| CD99 | mouse |
| Estrogen Receptor | mouse |
| p53 | mouse |
| Ep-CAM | mouse |
| CD 8 | mouse |
| Ki67 | mouse |
| PCNA | mouse |
| Keratin 13 human | mouse |
| Keratin 18 human | mouse |
| Keratin 14 human | mouse |
| Keratin 8 human | mouse |
| Keratin 17 human | mouse |

### Example 4

### Comparative Example

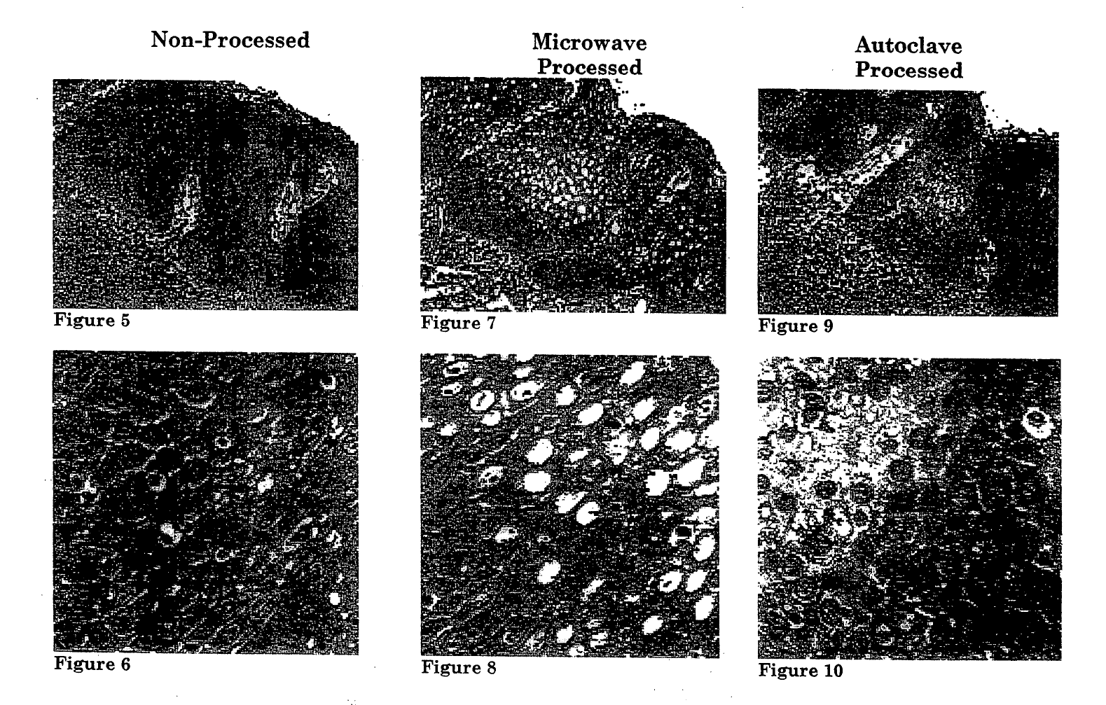

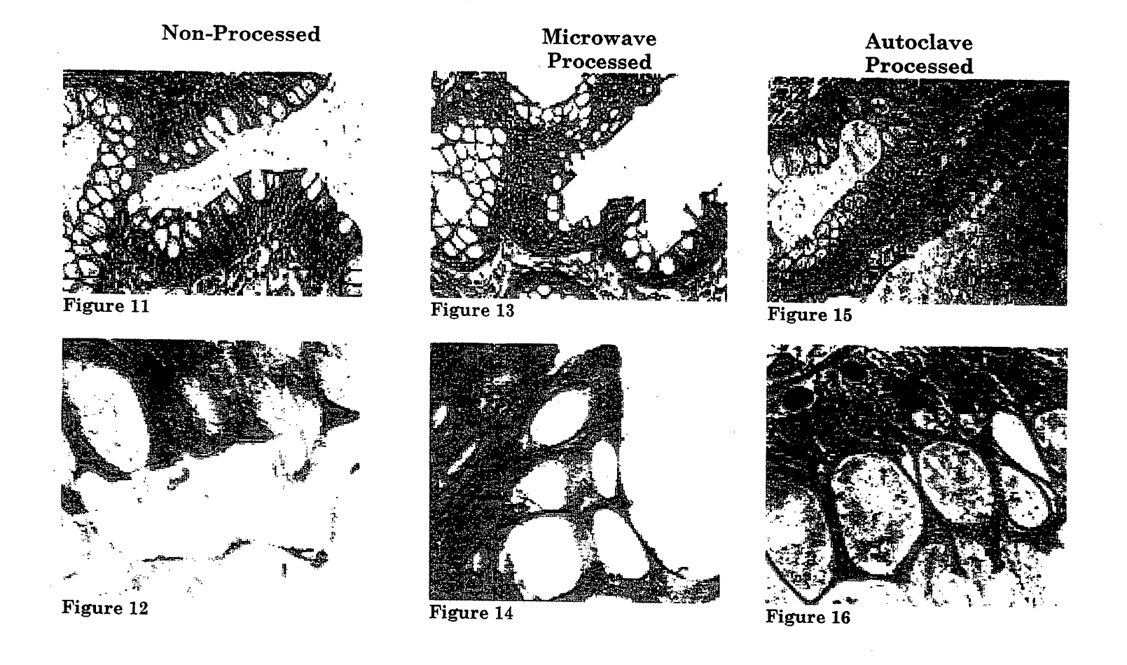

Due to thermal processing of the tissue by a method of the invention involving submersion of the sample and heating under pressure, only a limited number of gas bubbles form at the surface of the sections. This allows much better preservation of cell and tissue morphology in comparison to a treatment involving microwave boiling. In the present example, we performed comparative immunohistochemical staining of samples from the squamous epithelium of human cervix. FIGS. 5 through 10 represent the original unfixed tissue, formaldehyde fixed tissue processed by microwave treatment according to the prior art and formaldehyde fixed tissue processed by a method of the present invention, respectively. For comparison images of the tissue at all three treatments is presented at lower (FIGS. 5, 7 and 9) and higher (FIGS. 6, 8 and 10) magnification to compare the integrity of the tissue morphology after the treatment. Clearly in the microwave treatment loss of tissue morphology and holes in the tissue can be observed at the cellular level.

When the method for antigen retrieval according to the present invention was compared to autoclaving in a submersion fluid composition without glycerol, substantial morphological damage was observed in the samples treated in submersion fluid composition A of Table 1, whereas the use of submersion fluid compositions B and C resulted in essentially complete preservation of morphological characteristics.

Further, antigenicity could successfully be recovered from over-fixed tissue samples by the use of submersion fluid compositions comprising a guanidinium salt (submersion fluid compositions D, E, F, G and H in Table 1), although the use of submersion fluid compositions D revealed tissues of which the morphology was quite severely damaged.

### Example 5

### Immunofluorescent Staining

For immunofluorescent staining original samples were fixed with 3.6% buffered formaldehyde and washed in 3 changes of ethanol (70%;) for 24 hours and embedded, as described in Examples 1 and 2. Sections were processed as described for immunohistochemical staining in Example 3 and antigen retrieval was performed using submersion fluid compositions A, B and C of Table 1.

Staining was performed with a primary rabbit antibody to mouse keratin 5 (mouse skin) and with a mouse monoclonal antibody to human E-cadherin (Mob HECD-1) in a concentration of 1 μg/ml. The primary antibodies were detected with Goat anti-rabbit IgG labelled with Alexa 488 or Alexa 546 (both from Molecular Probes) or with Goat anti-Mouse IgG1 (PickCell Laboratories, Leiden, The Netherlands) conjugated with Cy5 (Amersham Biosciences, Freiburg, Germany). The samples were observed with Nicon Ecclips 800 equipped with suitable fluorescence filters.

A specific strong signal together with a low background fluorescence was observed in the tissue samples treated with all submersion fluid composition tested. However, the morphology of the samples was damaged in the case of submersion fluid compositions A.

When the same procedure was repeated with the use of microwave induced antigen retrieval instead of using the autoclave method, high background fluorescence was observed in all samples tested. It was therefore concluded that immunofluorescent staining of samples fixed by cross-linking agents is possible only when antigens are retrieved by the method of heating under pressure. When preservation of morphology is therefore to be ensured, immunofluorescent staining of samples fixed by cross-linking agents is suitably performed by a method of the present invention.

1. Method for the preparation of a sample, comprising (a) providing a sample comprising an antigen that has been fixed by a cross-linking agent with crosslinking of the antigen; (b) retrieving the antigen for immunological staining by submerging said sample in a submersion fluid composition comprising an osmotically active compound selected from the group consisting of polyols alditols, aldoses, methylamines, amino acids and derivatives and combinations thereof; and (c heating the submerged sample under pressure.

2. Method according to claim 1, wherein said heating under pressure comprises heating to a temperature of between 100° C. and 130° C. under a corresponding pressure of between 1.01 and 2.70 bara.

3. Method according to claim 2, wherein said heating under pressure comprises the heating to a temperature of about 121° C. under a pressure of about 2 bara.

4. Method according to claim 1, wherein said composition comprises between 1 and 99 wt. % of the osmotically active compound, based on the weight of the composition.

5. Method according to claim 4, wherein said composition comprises between 15 and 60 wt. % of the osmotically active compound, based on the weight of the composition.

6. (canceled)

7. Method according to claim 1, wherein said osmotically active compound is glycerol.

8. Method according to claim 1, wherein said fluid composition additionally comprises a guanidinium salt.

9. Method for the preparation of a sample, comprising (a) providing a sample comprising an antigen that has been fixed by a cross-linking agent with crosslinking of the antigen; (b) retrieving the antigen for immunofluorescent staining by submerging said sample in a submersion fluid composition comprising an osmotically active compound selected from the group consisting of the group consisting of polyols, alditols, aldoses, methylamines, amino acids and derivatives and combinations and (c) heating the sad submerged sample under pressure.

10. Method according to claim 9, wherein an autoclave is used for heating the submerged sample under pressure.

11. A submersion fluid composition for retrieving the antigenicity of samples fixed by cross-linking agents comprising an osmotically active compound.

12. A composition according to claim 11, comprising between 1 and 99 wt. % of an osmotically active compound, based on the weight of the composition.

13. A composition according to claim 12, comprising between 15 and 60 wt. % of an osmotically active compound, based on the weight of the composition.

14. A composition according to claim 11, wherein said osmotically active compound is selected from the group consisting of polyols, aldotils, aldoses, methylamines, amino acids or derivatives or combinations thereof.

15. A composition according to claim 14, wherein said osmotically active compound is glycerol.

16. A composition according to claim 11, further comprising a guanidinium salt.

17-19. (canceled)

20. Method according to claim 1, wherein an autoclave is used for heating the submerged sample under pressure.

21. Method according to claim 1, further comprising the step (d) of identifying the retrieved antigen by immunological staining.

22. Method according to claim 9, further comprising the step (d) of identifying the retrieved antigen by immunofluorescent staining.
